(19) United States
(12) Patent Application Publication
(10) Pub. No.: US 2024/0081709 A1
(43) Pub. Date: Mar. 14, 2024

(54) BIOLOGICAL INFORMATION MEASUREMENT DEVICE AND BIOLOGICAL INFORMATION MEASUREMENT METHOD

(71) Applicant: SHINKO ELECTRIC INDUSTRIES CO., LTD., Nagano-shi (JP)

(72) Inventor: Kazuhiro YOSHIDA, Nagano-shi (JP)

(21) Appl. No.: 18/459,906

(22) Filed: Sep. 1, 2023

(30) Foreign Application Priority Data
Sep. 8, 2022 (JP) 2022-143015

(51) Int. Cl. A61B 5/256; A61B 5/00

(52) U.S. Cl. CPC A61B 5/256; A61B 5/0006

(57) ABSTRACT

A biological information measurement device includes a sensor unit and a controller. The sensor unit includes an N number of measurement portions. N is a natural number of 3 or greater. The controller acquires an N number of output signals from the measurement portions in a given acquisition order. The controller outputs a shutdown signal having a first level to the measurement portions in accordance with the acquisition order of the output signals. The controller outputs the shutdown signal having a second level to the measurement portions in response to acquisition of a corresponding output signal. When the controller outputs the first-level shutdown signal to the measurement portion having an ordinal number of i in the acquisition order, where i is a natural number of 1 or greater, the controller also outputs the first-level shutdown signal to the measurement portion having an ordinal number of (i+1) in the acquisition order.

### CROSS-REFERENCE TO RELATED APPLICATIONS

This application is based upon and claims the benefit of priority from prior Japanese Patent Application No. 2022-143015, filed on Sep. 8, 2022, the entire contents of which are incorporated herein by reference.

### FIELD

This disclosure relates to a biological information measurement device and a biological information measurement method.

### BACKGROUND

A biological information measurement device (sensor device) is an electronic device attached to an individual, or subject, to measure a signal corresponding to biological information of the subject. Japanese Laid-Open Patent Publication No. 2011-56243 describes one example of such a biological information measurement device. The biological information measurement device includes a sensor and a controller. The sensor detects an electrical signal corresponding to the biological information of the subject. The controller performs signal processing on the electrical signal detected by the sensor.

### SUMMARY

Biological information measurement devices are required to consume less power.

This Summary is provided to introduce a selection of concepts in a simplified form that are further described below. This Summary is not intended to identify key features or essential features of the claimed subject matter, nor is it intended to be used as an aid in determining the scope of the claimed subject matter.

One embodiment of a biological information measurement device includes a sensor unit and a controller. The sensor unit includes an N number of measurement portions, where N is a natural number of 3 or greater. The controller is configured to sequentially acquire an N number of output signals from the N number of measurement portions in a given acquisition order. Each of the N number of measurement portions includes a biological electrode and an amplifier. The amplifier is configured to amplify biological information measured by the biological electrode to generate the output signal. The amplifier includes a shutdown terminal configured to receive a shutdown signal. The amplifier is configured to operate in a drive mode to generate the output signal in response to the shutdown signal having a first level and operate in a shutdown mode to suspend generation of the output signal in response to the shutdown signal having a second level. The controller is configured to sequentially output the shutdown signal having the first level to the N number of measurement portions in accordance with the acquisition order of the N number of output signals. The controller is configured to output the shutdown signal having the second level to each of the N number of measurement portions in response to acquisition of a corresponding one of the output signals. When the controller outputs the shutdown signal having the first level to the one of the measurement portions having an ordinal number of i in the acquisition order, where i is a natural number of 1 or greater, the controller is configured to also output the shutdown signal having the first level to the one of the measurement portions having an ordinal number of (i+1) in the acquisition order.

Other features and aspects will be apparent from the following description, the drawings, and the claims.

### BRIEF DESCRIPTION OF THE DRAWINGS

The embodiments, together with objects and advantages thereof, may best be understood by reference to the following description of the presently preferred embodiments together with the accompanying drawings in which:

FIG. 1 is a schematic block diagram illustrating one embodiment of a biological information measurement system;

FIG. 2 is a perspective view schematically illustrating the biological information measurement system of FIG. 1 in use;

FIGS. 3, 4, 5, 6, and 7 are schematic diagrams illustrating one embodiment of a method for measuring biological information;

FIG. 8 is a perspective view schematically illustrating a modified example of the biological information measurement system in use; and

FIG. 9 is a perspective view schematically illustrating another modified example of the biological information measurement system in use.

### DESCRIPTION OF THE EMBODIMENTS

One embodiment will now be described with reference to the drawings. In the accompanying drawings, elements are illustrated for simplicity and clarity and have not necessarily been drawn to scale.

Overall Configuration of Biological Information Measurement System 1

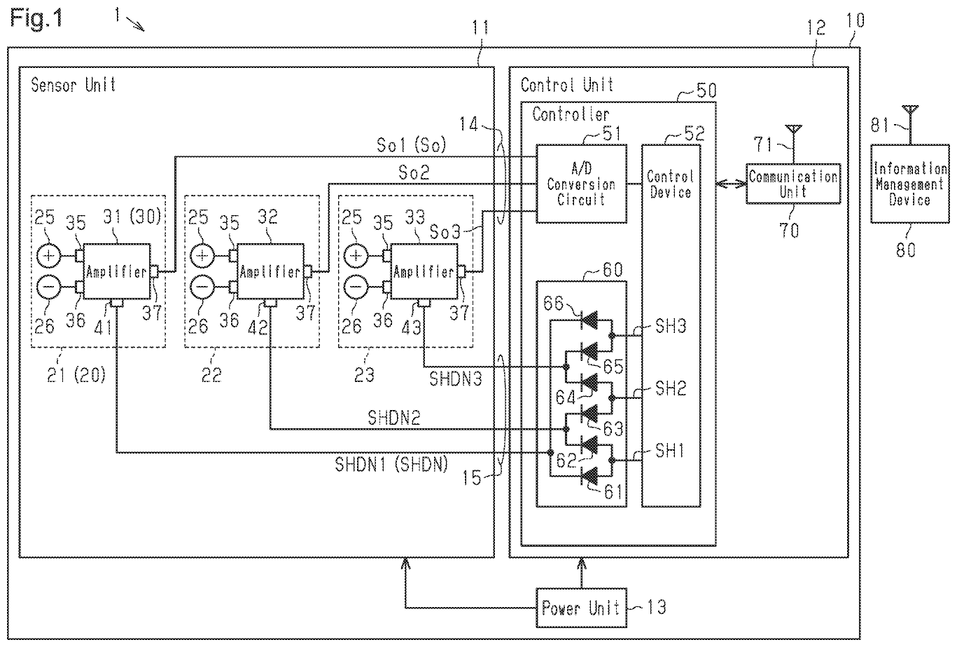

As illustrated in FIG. 1, a biological information measurement system 1 includes a biological information measurement device 10 and an information management device 80.

Configuration of Biological Information Measurement Device 10

The biological information measurement device 10 includes, for example, a sensor unit 11, a control unit 12, and a power unit 13. The biological information measurement device 10 includes, for example, a first signal line 14, which transmits a signal output from the sensor unit 11 to the control unit 12, and a second signal line 15, which transmits a signal output from the control unit 12 to the sensor unit 11.

The power unit 13 is configured to, for example, supply the sensor unit 11 and the control unit 12 with electric power used to operate the sensor unit 11 and the control unit 12. The power unit 13 may be, for example, incorporated in the sensor unit 11 or the control unit 12 or arranged outside the sensor unit 11 and the control unit 12. The power unit 13 may be, for example, a button cell or a coin battery. In the present embodiment, the power unit 13 is a button cell incorporated in the control unit 12.

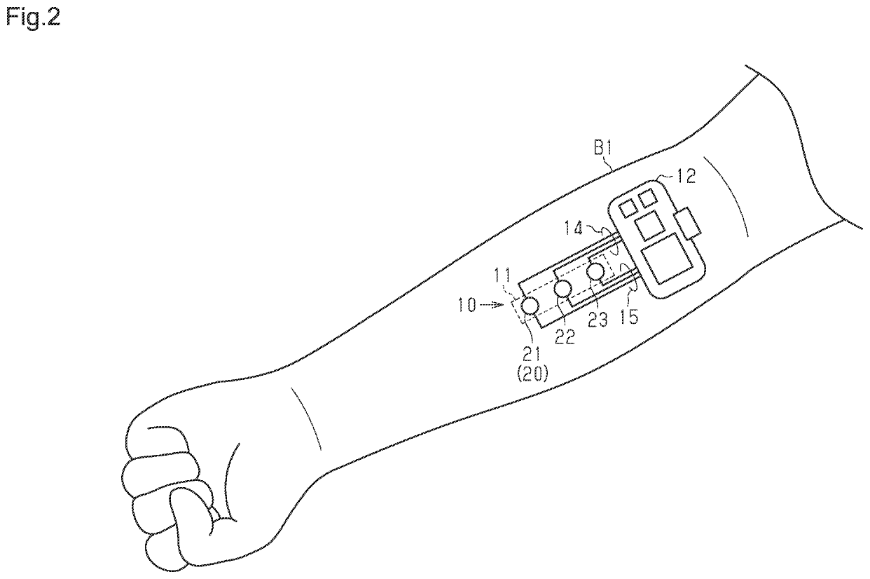

As illustrated in FIG. 2, the biological information measurement device 10 is attached to, for example, a body B1 of an individual, or subject. The biological information measurement device 10 is configured to acquire biological information of the subject. The body B1 may be, for example, an arm or leg of the subject. In the present embodiment, the body B1 is the forearm of the subject. The biological information is, for example, biopotentials. Biopotentials may be any potential that varies and indicates biological information. Examples of biopotentials include electrocardiograms, electromyograms, electroencephalograms, and heart rates. In the present embodiment, the biological information measurement device 10 is configured to measure myoelectric signals indicating myogenic potentials of the subject produced during contractile activity of the muscle fibers (muscle). In the present embodiment, the biological information measurement device 10 is configured to measure myoelectric signals at multiple parts (multiple locations) of the subject's body B1. In the present embodiment, the sensor unit 11 is a myoelectric sensor that detects myoelectric signals corresponding to myogenic potentials.

Configuration of Sensor Unit 11

The sensor unit 11 includes an N number of channels, where N is a natural number of 3 or greater. In correspondence with the N number of (in this case, three) channels, the sensor unit 11 includes an N number (in this case, three) of measurement portions 21, 22, and 23. The three measurement portions 21, 22, and 23 are attached to, for example, different parts of the subject's body B1. That is, the sensor unit 11 includes the measurement portions 21, 22, and 23 arranged to measure myogenic potentials at different measuring parts. For example, when the sensor unit 11 is attached to the forearm of the individual, the three measurement portions 21, 22, and 23 are attached to the subject arranged on the forearm in the longitudinal direction of the forearm. The measurement portions 21, 22, and 23 each measure the myogenic potential at the part where it is attached and outputs a myoelectric signal corresponding to the myogenic potential. The three measurement portions 21, 22, and 23 have the same structure. Thus, only the measurement portion 21 will be described in detail. The measurement portions 22 and 23 will not be described in detail.

As illustrated in FIG. 1, the measurement portion 21 includes, for example, two biological electrodes 25 and 26 and an amplifier 31. Although not illustrated in the drawings, the measurement portion 21 for example, has the two biological electrodes 25 and 26 arranged on one side of a board and the amplifier 31 arranged on the other side of the board. The two biological electrodes 25 and 26 are arranged to contact the body B1 (refer to FIG. 2) of the subject.

The two biological electrodes 25 and 26 includes the positive biological electrode 25 and the negative biological electrode 26. The two biological electrodes 25 and 26 measure the myogenic potential at where the biological electrodes 25 and 26 are attached. The myogenic potential measured by the biological electrodes 25 and 26 is a weak potential of about several microvolts to several millivolts. The myogenic potential is in a frequency band of about 5 Hz to 500 Hz.

The amplifier 31 includes two input terminals 35 and 36, one output terminal 37, and one shutdown terminal 41. The two input terminals 35 and 36 are connected to the two biological electrodes 25 and 26, respectively. The two input terminals 35 and 36 receive the myogenic potential measured by the two biological electrodes 25 and 26. The output terminal 37 is connected by the first signal line 14 to the control unit 12. In other words, the first signal line 14 connects the output terminal 37 of the amplifier 31 and the control unit 12. The shutdown terminal 41 is connected by the second signal line 15 to the control unit 12. In other words, the second signal line 15 connects the shutdown terminal 41 of the amplifier 31 and the control unit 12. The amplifier 31 is, for example, a differential amplifier.

The amplifier 31 generates an output signal So1 that is a myoelectric signal obtained by amplifying the weak myogenic potential input to the two biological electrodes 25 and 26. The amplifier 31 outputs the output signal So1 through the first signal line 14 to the control unit 12. The output signal So1 of the amplifier 31 is an analog signal. The shutdown terminal 41 of the amplifier 31 receives a shutdown signal SHDN1 generated by the control unit 12. When the shutdown signal SHDN1 received by the shutdown terminal 41 has a high level (first level), the amplifier 31 operates in a drive mode and generates the output signal So1. When the shutdown signal SHDN1 received by the shutdown terminal 41 has a low level (second level), the amplifier 31 operates in a shutdown mode and suspends generation of the output signal So1.

The measurement portion 22 includes, for example, two biological electrodes 25 and 26 and an amplifier 32. The amplifier 32 includes two input terminals 35 and 36, one output terminal 37, and one shutdown terminal 42. The amplifier 32 generates an output signal So2 that is a myoelectric signal obtained by amplifying the weak myogenic potential input to the two biological electrodes 25 and 26. The amplifier 32 outputs the output signal So2 through the first signal line 14 to the control unit 12. The shutdown terminal 42 of the amplifier 32 receives a shutdown signal SHDN2 generated by the control unit 12. The amplifier 32 operates in a drive mode when the shutdown signal SHDN2 has a high level and operates in a shutdown mode when the shutdown signal SHDN2 has a low level.

The measurement portion 23 includes, for example, two biological electrodes 25 and 26 and an amplifier 33. The amplifier 33 includes two input terminals 35 and 36, one output terminal 37, and one shutdown terminal 43. The amplifier 33 generates an output signal So3 that is a myoelectric signal obtained by amplifying the weak myogenic potential input to the two biological electrodes 25 and 26. The amplifier 33 outputs the output signal So3 through the first signal line 14 to the control unit 12. The shutdown terminal 43 of the amplifier 33 receives a shutdown signal SHDN3 generated by the control unit 12. The amplifier 33 operates in a drive mode when the shutdown signal SHDN3 has a high level and operates in a shutdown mode when the shutdown signal SHDN3 has a low level.

Configuration of Control Unit 12

The control unit 12 includes, for example, a controller 50 and a communication unit 70. The controller 50 is, for example, electrically connected to three first signal lines 14 and to three second signal lines 15. The controller 50 is electrically connected to the communication unit 70.

The controller 50 includes, for example, an A/D conversion circuit 51 that converts an analog signal to a digital signal, a control device 52, and a shutdown signal generation circuit 60 that generates the shutdown signals SHDN1, SHDN2, and SHDN3.

The A/D conversion circuit 51 is, for example, electrically connected to the three first signal lines 14. The A/D conversion circuit 51 acquires an N number of output signals So1, So2, and So3 (analog signals) output from an N number of measurement portions 21, 22, and 23 at a given sampling rate and converts the acquired analog signals to digital signals. The A/D conversion circuit 51 acquires the N number of output signals So1, So2, and So3 in a given acquisition order. The A/D conversion circuit 51, for example, sequentially acquires the N number of output signals So1, So2, and So3 in an order of 1→2→ . . . →N→1→2 . . . . The A/D conversion circuit 51 sequentially acquires the N number of (in this case, three) output signals So1, So2, and So3 in the order of So1→So2→So3→So1→So2 . . . . Thus, in the A/D conversion circuit 51, the output signal So1 of the measurement portion 21 is obtained first, the output signal So2 of the measurement portion 22 is obtained second, and the output signal So3 of the measurement portion 23 is obtained third. Further, in the A/D conversion circuit 51, after the Nth (in this case, third) output signal So3 is obtained, the acquisition of signals is repeated in the order of So1→So2→So3. In this manner, the first to Nth output signals that are the N number of output signals are repetitively acquired in the order of the first output signal, the second output signal, . . . , the Nth output signal, the first output signal, the second output signal, . . . . The A/D conversion circuit 51 outputs the biological information (here, myoelectric signal), which has been converted into digital signals, to the control device 52. The sampling rate (sampling frequency) of the A/D conversion circuit 51 may be, for example, approximately 1 kHz.

In the description hereafter, to aid understanding, the output signal So1 may be referred to as the first output signal So1, the output signal So2 may be referred to as the second output signal So2, and the output signal So3 may be referred to as the third output signal So3. The measurement portion 21 that outputs the first output signal So1 may be referred to as the first measurement portion 21, the measurement portion 22 that outputs the second output signal So2 may be referred to as the second measurement portion 22, and the measurement portion 23 that outputs the third output signal So3 may be referred to as the third measurement portion 23. The amplifier 31 that outputs the first output signal So1 may be referred to as the first amplifier 31, the amplifier 32 that outputs the second output signal So2 may be referred to as the second amplifier 32, and the amplifier 33 that outputs the third output signal So3 may be referred to as the third amplifier 33. The shutdown terminal 41 of the amplifier 31 may be referred to as the first shutdown terminal 41, the shutdown terminal 42 of the amplifier 32 may be referred to as the second shutdown terminal 42, and the shutdown terminal 43 of the amplifier 33 may be referred to as the third shutdown terminal 43. The output signals So1, So2, and So3 will be referred to collectively as the output signals So, the measurement portions 21, 22, and 23 will be referred to collectively as the measurement portions 20, and the amplifiers 31, 32, and 33 will be referred to collectively as the amplifiers 30.

The control device 52 is configured to, for example, centrally control the operation of each circuit included in the controller 50. The control device 52, for example, generates signals for controlling the order in which the A/D conversion circuit 51 acquires the output signals So1, So2, and So3 and outputs the signals to the A/D conversion circuit 51. The control device 52, for example, conducts a given analyzing processing on the digital signals generated by the A/D conversion circuit 51, that is, the biological information (here, myoelectric signals), and generates analysis result information. The control device 52, for example, outputs the digital signals generated by the A/D conversion circuit 51, that is, the biological information (here, myoelectric signals), or the analysis result information to the communication unit 70.

The control device 52 generates control signals SH1, SH2, and SH3 that respectively control the operation modes of the three amplifiers 31, 32, and 33 and outputs the control signals SH1, SH2, and SH3 to the shutdown signal generation circuit 60. The control signal SH1 controls the operation mode of the first amplifier 31. The control signal SH2 controls the operation mode of the second amplifier 32. The control signal SH3 controls the operation mode of the third amplifier 33. In the description hereafter, to aid understanding, the control signal SH1 may be referred to as the first control signal SH1, the control signal SH2 may be referred to as the second control signal SH2, and the control signal SH3 may be referred to as the third control signal SH3.

The control device 52, for example, sequentially switches the control signals SH1, SH2, and SH3 from a low level to a high level to operate the N number of amplifiers 31, 32, and 33 in the drive mode in accordance with an acquisition order of the N number of output signals So1, So2, and So3. The control device 52, for example, sequentially switches the control signals SH1, SH2, and SH3 from a low level to a high level in the order of the control signal SH1, the control signal SH2, and the control signal SH3. In response to acquisition of the output signals So1, So2, and So3, the control device 52, for example, switches the corresponding control signals SH1, SH2, and SH3 from the high level to the low level to operate the corresponding amplifiers 31, 32, and 33 in the shutdown mode. For example, after the A/D conversion circuit 51 completes acquisition of the first output signal So1, the control device 52 switches the first control signal SH1 from the high level to the low level. For example, after the A/D conversion circuit 51 completes acquisition of the second output signal So2, the control device 52 switches the second control signal SH2 from the high level to the low level. For example, after the A/D conversion circuit 51 completes acquisition of the third output signal So3, the control device 52 switches the third control signal SH3 from the high level to the low level.

The signals that control the acquisition order of the output signals So1, So2, and So3 in the A/D conversion circuit 51 may be the same as or differ from the control signals SH1, SH2, and SH3.

The control device 52 may be configured as circuitry including 1) one or more processors that run on computer programs (software) to execute various processes, 2) one or more dedicated hardware circuits such as application-specific integrated circuits (ASICs) that execute at least some of the processes, or 3) a combination of processors and hardware circuits. Each processor includes a central processing unit (CPU), a random access memory (RAM), a read only memory (ROM), and the like. The memory stores program codes or instructions configured to have the CPU execute processes. The memory, namely, a computer readable medium, includes any available medium that is accessible by a versatile or dedicated computer.

The shutdown signal generation circuit 60 generates the shutdown signals SHDN1, SHDN2, and SHDN3 based on the control signals SH1, SH2, and SH3 received from the control device 52. In the description hereafter, the shutdown signals SHDN1, SHDN2, and SHDN3 will be referred to collectively as the shutdown signals SHDN.

The shutdown signal generation circuit 60 includes, for example, a plurality of (here, six) the diodes 61, 62, 63, 64, 65, and 66.

The anode terminals of the diodes 61 and 62 receive the control signal SH1. The anode terminals of the diodes 63 and 64 receive the control signal SH2. The anode terminals of the diodes 65 and 66 receive the control signal SH3.

The cathode terminal of the diode 61 is connected to the cathode terminal of the diode 66. The cathode terminals of the diodes 61 and 66 are connected by the second signal line 15 to the shutdown terminal 41 of the amplifier 31 in the measurement portion 21.

The cathode terminal of the diode 62 is connected to the cathode terminal of the diode 63. The cathode terminals of the diodes 62 and 63 are connected by the second signal line 15 to the shutdown terminal 42 of the amplifier 32 in the measurement portion 22.

The cathode terminal of the diode 64 is connected to the cathode terminal of the diode 65. The cathode terminals of the diodes 64 and 65 are connected by the second signal line 15 to the shutdown terminal 43 of the amplifier 33 in the measurement portion 23.

The first control signal SH1 is input via the diode 61 as the shutdown signal SHDN1 to the first amplifier 31, and input via the diode 62 as the shutdown signal SHDN2 to the second amplifier 32.

The second control signal SH2 is input via the diode 63 as the shutdown signal SHDN2 to the second amplifier 32, and input via the diode 64 as the shutdown signal SHDN3 to the third amplifier 33.

The third control signal SH3 is input via the diode 65 as the shutdown signal SHDN3 to the third amplifier 33, and input via the diode 66 as the shutdown signal SHDN1 to the first amplifier 31.

A high-level shutdown signal SHDN has a higher voltage than a low-level shutdown signal SHDN. Further, the high-level control signals SH1, SH2, and SH3 have a higher voltage than the low-level control signals SH1, SH2, and SH3. Thus, in the shutdown signal generation circuit 60, for example, when one of the two control signals SH1 and SH3 has a high level and the other one has a low level, priority is given to the control signal having the high level. For example, in the diodes 61 and 66 of the shutdown signal generation circuit 60, when one of the two control signals SH1 and SH3 has a high level, priority is given to the control signal having the high level, and the shutdown signal SHDN1 is set to the high level. For example, in the diodes 62 and 63 of the shutdown signal generation circuit 60, when one of the two control signals SH1 and SH2 has a high level, priority is given to the control signal having the high level, and the shutdown signal SHDN2 is set to the high level. For example, in the diodes 64 and 65 of the shutdown signal generation circuit 60, when one of the two control signals SH2 and SH3 has a high level, priority is given to the control signal having the high level, and the shutdown signal SHDN3 is set to the high level.

When the control signal SH1 has a high level, the shutdown signal SHDN1 input to the first amplifier 31 is set to a high level, and the shutdown signal SHDN2 input to the second amplifier 32 is set to a high level. When the control signal SH2 has a high level, the shutdown signal SHDN2 input to the second amplifier 32 is set to a high level, and the shutdown signal SHDN3 input to the third amplifier 33 is set to a high level. When the control signal SH3 has a high level, the shutdown signal SHDN3 input to the third amplifier 33 is set to a high level, and the shutdown signal SHDN1 input to the first amplifier 31 is set to a high level.

In this manner, in the shutdown signal generation circuit 60, when a jth control signal has a high level, where j is a natural number of 1 to N−1, the jth shutdown signal SHDN is set to a high level, and the (j+1)th shutdown signal SHDN is set to a high level. In other words, when the shutdown signal generation circuit 60 outputs a shutdown signal SHDN at a high level to the jth measurement portion 20, the shutdown signal generation circuit 60 also outputs a shutdown signal SHDN at a high level to the (j+1)th measurement portion 20. For example, when the shutdown signal generation circuit 60 outputs the shutdown signal SHDN1 at a high level to the first measurement portion 21, the shutdown signal generation circuit 60 also outputs the shutdown signal SHDN2 at a high level to the second measurement portion 22. For example, when the shutdown signal generation circuit 60 outputs the shutdown signal SHDN2 at a high level to the second measurement portion 22, the shutdown signal generation circuit 60 also outputs the shutdown signal SHDN2 at a high level to the third measurement portion 23. Further, in the shutdown signal generation circuit 60, when the Nth (here, third) control signal SH3 has a high level, the Nth shutdown signal SHDN3 is shifted to a high level, and the first shutdown signal SHDN1 is shifted to a high level. In other words, when the shutdown signal generation circuit 60 outputs the shutdown signal SHDN3 at a high level to the Nth (here, third) measurement portion 23, the shutdown signal generation circuit 60 also outputs the shutdown signal SHDN1 at a high level to the first measurement portion 23.

In the shutdown signal generation circuit 60, when the two control signals SH1 and SH3 both shift to a low level, the shutdown signal SHDN1 is shifted from a high level to a low level. In the shutdown signal generation circuit 60, when the two control signals SH1 and SH2 both shift to a low level, the shutdown signal SHDN2 is shifted from a high level to a low level. In the shutdown signal generation circuit 60, when the two control signals SH2 and SH3 both shift to a low level, the shutdown signal SHDN3 is shifted from a high level to a low level.

As illustrated in FIG. 2, the N number of measurement portions 21, 22, and 23 are sequentially arranged in a first direction in an order corresponding to the acquisition order of the output signals So1, So2, and So3. In the example of FIG. 2, the first direction corresponds to the longitudinal direction of the arm, which is the body B1 of the subject, and extends from the wrist toward the upper arm. In the sensor unit 11 of the present embodiment, the N number of measurement portions 21, 22, and 23 are sequentially arranged from a position closer to the subject's wrist to a position closer to the subject's upper arm in an order corresponding to the acquisition order of the output signals So1, So2, and So3, that is, in the order of the measurement portion 21, the measurement portion 22, and the measurement portion 23. In the example of FIG. 2, the first measurement portion 21 is located at the position that is the closest to the wrist of the subject, the second measurement portion 22 is located toward the upper arm from the measurement portion 21, and the third measurement portion 23 is located toward the upper arm from the measurement portion 22.

Configuration of Communication Unit 70

As illustrated in FIG. 1, the communication unit 70 is connected to an antenna 71 to perform communication with the information management device 80 in accordance with a given wireless communication protocol. The communication unit 70 is, for example, a transmission circuit. The communication unit 70 sends transmission information, which includes the biological information (here, myoelectric signal) and analysis result information acquired by the sensor unit 11, to the antenna 71. The communication unit 70 sends the transmission information with the antenna 71 through wireless communication to the information management device 80. Examples of wireless communication protocols include Bluetooth® Low Energy (BLE), ZigBee®, ANT+®, NFC, and the like.

Configuration of Information Management Device 80

The information management device 80 includes, for example, an antenna 81 and receives the information sent from the biological information measurement device 10. The information management device 80, for example, stores the received information in a storage device. The storage device may be, for example, a hard disk drive (HDD). The information management device 80, for example, displays the received information on a display device. The information management device 80, for example, performs a given analyzing processing on the received information and displays the analysis result on the display device. The display device may be, for example, a liquid crystal display or an organic electroluminescent (EL) display. The information management device 80, for example, issues a notification of the received information through a given action using sound or vibration. Sound may be, for example, sound that expresses words with a voice, sound such as a bell ring that does not express words, and a combination of such sounds.

The information management device 80 may be attached together with the biological information measurement device 10 to the subject's body B1 (refer to FIG. 2) or located at a position separated from the body B1.

Method for Measuring Biological Information

A method for measuring biological information with the biological information measurement device 10 will now be described.

As illustrated in FIG. 2, the biological information measurement device 10 is attached to the body B1 of the subject, in this case, the forearm. The biological information measurement device 10 is attached to the body B1 so that the biological electrodes 25 and 26 of each of the N number of measurement portions 21, 22, and 23 (refer to FIG. 1) are in contact with the skin of the body B1.

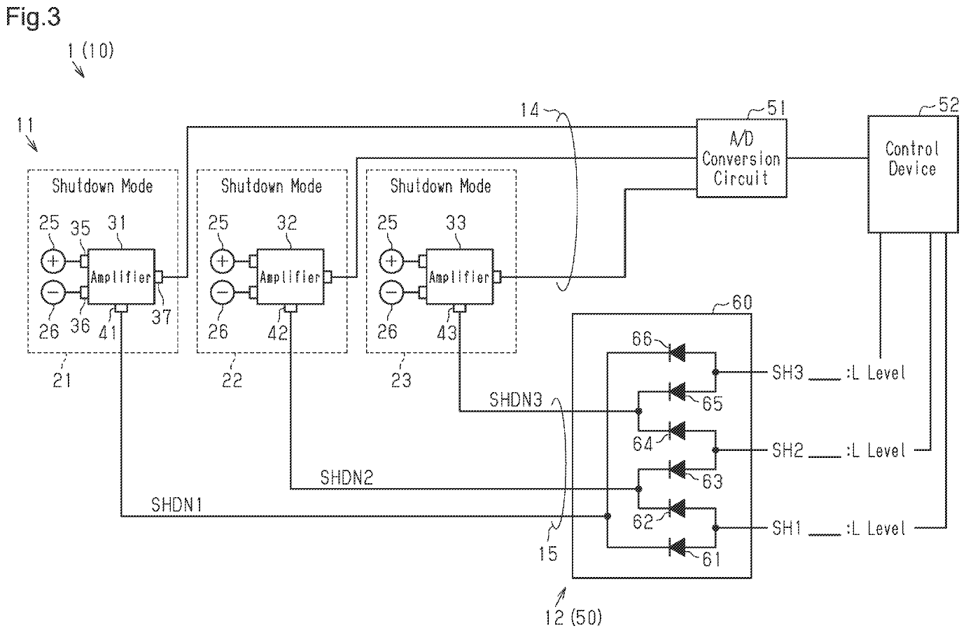

Then, referring to FIG. 3, the biological information measurement device 10 initiates biological information measurement. When initiating measurements, the N number of control signals SH1, SH2, and SH3 all have a low level, and the N number of shutdown signals SHDN1, SHDN2, and SHDN3 all have a low level. Thus, the N number of amplifiers 31, 32, and 33 are all in the shutdown mode.

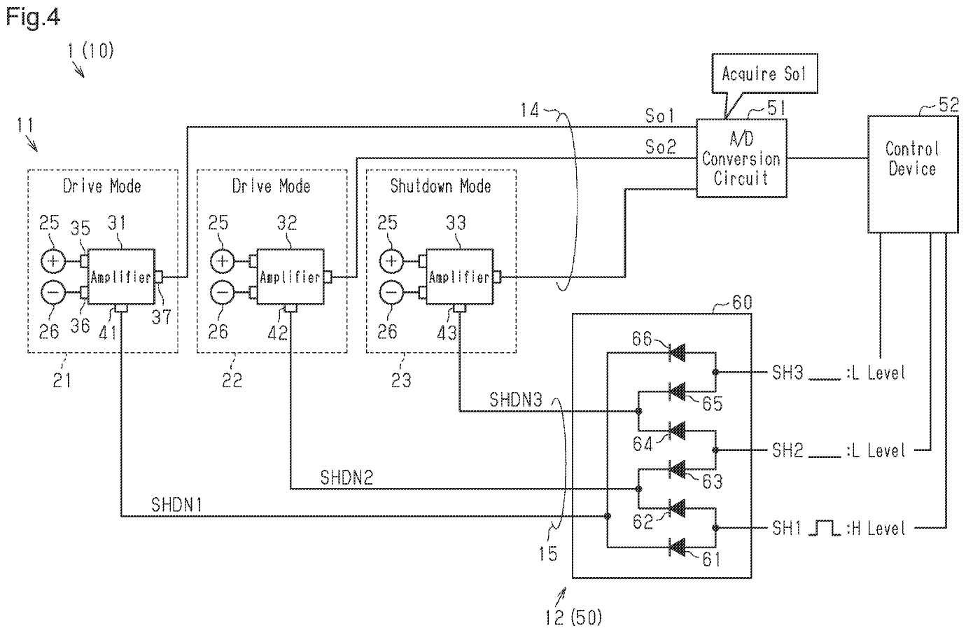

With reference to FIG. 4, in accordance with the acquisition order of the output signals So1, So2, and So3, the control device 52 first shifts the first control signal SH1 from the low level to a high level. When receiving the high-level control signal SH1, the shutdown signal generation circuit 60 outputs the shutdown signal SHDN1 at a high level and outputs the shutdown signal SHDN2 at a high level. This switches the amplifier 31 that receives the high-level shutdown signal SHDN1 to the drive mode and switches the amplifier 32 that receives the high-level shutdown signal SHDN2 to the drive mode. Then, the control device 52 waits for at least a switching time to allow the amplifiers 31, 32, and 33 to switch from the shutdown mode to the drive mode. The time period for switching the amplifiers 31, 32, and 33 from the shutdown mode to the drive mode is about 100 μs to 200 μs.

The amplifier 31, when switched to the drive mode, amplifies the myogenic potential measured by the biological electrodes 25 and 26 to generate the output signal So1 and outputs the output signal So1 to the A/D conversion circuit 51 of the controller 50. In the same manner, the amplifier 32, when switched to the drive mode, amplifies the myogenic potential measured by the biological electrodes 25 and 26 to generate the output signal So2 and outputs the output signal So2 to the A/D conversion circuit 51. Further, among the output signals So1 and So2, the A/D conversion circuit 51 acquires the output signal So1 that is the first one in the acquisition order. For example, in response to the high level control signal SH1, the A/D conversion circuit 51 acquires the output signal So1 at a given sampling rate. The sampling rate of the A/D conversion circuit 51 may be approximately 1 kHz. The A/D conversion circuit 51 converts the acquired output signal So1 (analog signal) into a digital signal and outputs the converted digital signal to the control device 52. The control device 52, for example, performs a given analyzing processing on the digital signal received from the A/D conversion circuit 51. This acquires biological information (here, myoelectric signal) as a digital signal at the part where the first measurement portion 21 is attached.

In the step illustrated in FIG. 4, the control signals SH2 and SH3 have a low level. Thus, the shutdown signal SHDN3 input to the third amplifier 33 has a low level. This maintains the amplifier 33 in the shutdown mode. Thus, the amplifier 33 is set in a quiescent state during the period the first output signal So1 is being acquired. Consequently, the amplifier 33 consumes less power than when the amplifier 33 is in the drive mode.

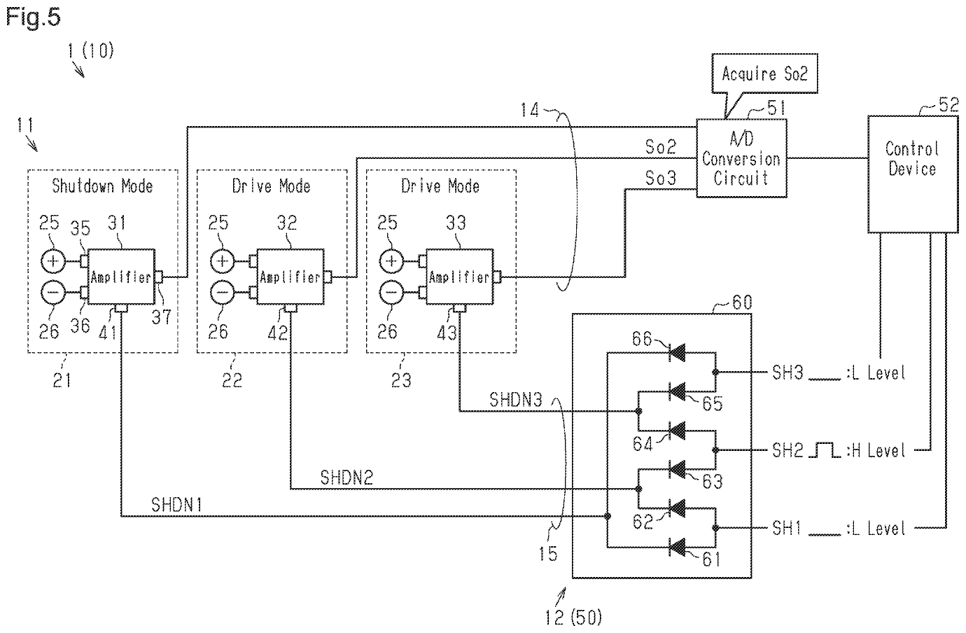

Then, referring to FIG. 5, in accordance with the acquisition order of the output signals So1, So2, and So3, the control device 52 shifts the second control signal SH2 from the low level to a high level. When receiving the high level control signal SH2, the shutdown signal generation circuit 60 outputs the shutdown signal SHDN2 at a high level and outputs the shutdown signal SHDN3 at a high level. This continues to operate the amplifier 32 that receives the high-level shutdown signal SHDN2 in the drive mode and switches the amplifier 33 that receives the high-level shutdown signal SHDN3 to the drive mode. In this state, the second amplifier 32 has been switched together with the first amplifier 31 to the drive mode when the amplifier 31 was switched to the drive mode (refer to FIG. 4). Thus, the amplifier 32 continues to operate in the drive mode in accordance with the high-level shutdown signal SHDN2. When switching the mode of the first amplifier 31 during measurement initiation, there is a need to wait for the switching time to allow for switching from the shutdown mode to the drive mode. In this respect, the second amplifier 32 has been switched to the drive mode together with the first amplifier 31 and is already operating in the drive mode when the second control signal SH2 shifts from the low level to the high level. Thus, after the second control signal SH2 shifts to the high level, the amplifier 32 operates in the drive mode without waiting for the switching time to allow for switching from the shutdown mode to the drive mode.

The amplifier 32, which is operating in the drive mode, continues to output the output signal So2 to the A/D conversion circuit 51. The amplifier 33, which has been switched to the drive mode, amplifies the myogenic potential measured by the biological electrodes 25 and 26 to generate the output signal So3 and outputs the output signal So3 to the A/D conversion circuit 51. Further, among the output signals So2 and So3, the A/D conversion circuit 51 acquires the output signal So2 that is the second one in the acquisition order. For example, in response to the high level the control signal SH2, the A/D conversion circuit 51 acquires the output signal So2 at a given sampling rate. The A/D conversion circuit 51 converts the acquired output signal So2 (analog signal) into a digital signal and outputs the converted digital signal to the control device 52. The control device 52, for example, performs a given analyzing processing on the digital signal received from the A/D conversion circuit 51. This acquires biological information (here, myoelectric signal) as a digital signal at the part where the second measurement portion 22 is attached.

In the step illustrated in FIG. 5, in response to acquisition of the output signal So1, the control device 52 shifts the first control signal SH1 from the high level to the low level. For example, after the A/D conversion circuit 51 completes acquisition of the output signal So1, the control device 52 shifts the control signal SH1 to the low level. Since the control signals SH1 and SH3 both have a low level, the shutdown signal SHDN1 is shifted to a low level. In response to the low-level shutdown signal SHDN1, the amplifier 31 is switched from the drive mode to the shutdown mode. Thus, the amplifier 31 is set in a quiescent state during the period the second output signal So2 is being acquired. Consequently, the amplifier 31 consumes less power than when the amplifier 31 remains operating in the drive mode.

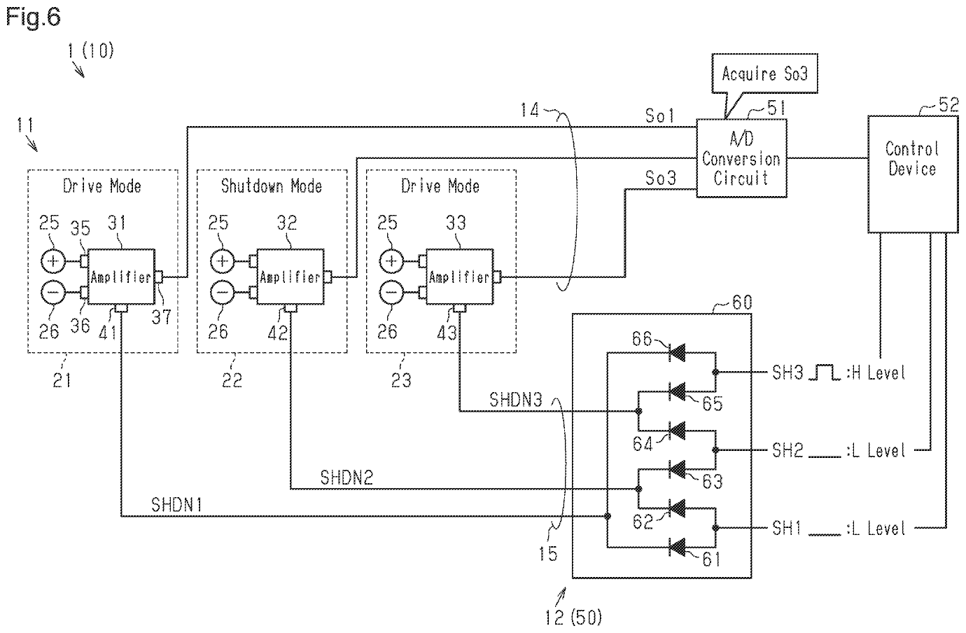

Then, referring to FIG. 6, in accordance with the acquisition order of the output signals So1, So2, and So3, the control device 52 shifts the third control signal SH3 from the low level to a high level. When receiving the high level control signal SH3, the shutdown signal generation circuit 60 outputs the shutdown signal SHDN3 at a high level and outputs the shutdown signal SHDN1 at a high level. This continues to operate the amplifier 33 that receives the high-level shutdown signal SHDN3 in the drive mode and switches the amplifier 31 that receives the high-level shutdown signal SHDN1 to the drive mode. In this state, the third amplifier 33 has been switched together with the second amplifier 32 to the drive mode when the amplifier 32 was switched to the drive mode (refer to FIG. 5). Thus, the amplifier 33 has been operating in the drive mode before the control signal SH3 shifts from the low level to the high level. Thus, after the third control signal SH3 shifts to the high level, the amplifier 33 operates in the drive mode without waiting for the switching time to allow for switching from the shutdown mode to the drive mode.

The amplifier 33, which is operating in the drive mode, continues to output the output signal So3 to the A/D conversion circuit 51. The amplifier 31, which has been switched to the drive mode, amplifies the myogenic potential measured by the biological electrodes 25 and 26 to generate the output signal So1 and output the output signal So1 to the A/D conversion circuit 51. Further, among the output signals So3 and So1, the A/D conversion circuit 51 outputs the output signal So3 that is the third (Nth) one in the acquisition order. For example, in response to the high level the control signal SH3, the A/D conversion circuit 51 acquires the output signal So3 at a given sampling rate. The A/D conversion circuit 51 converts the acquired output signal So3 (analog signal) into a digital signal and outputs the converted digital signal to the control device 52. The control device 52, for example, performs a given analyzing processing on the digital signal received from the A/D conversion circuit 51. This acquires biological information (here, myoelectric signal) as a digital signal at the part where the third measurement portion 23 is attached.

In the step illustrated in FIG. 6, in response to acquisition of the output signal So2, the control device 52 shifts the second control signal SH2 from the high level to a low level. For example, after the A/D conversion circuit 51 completes acquisition of the output signal So2, the control device 52 shifts the control signal SH2 to the low level. Thus, the control signals SH1 and SH2 both have a low level. This shifts the shutdown signal SHDN2 to the low level. In response to the low-level shutdown signal SHDN2, the amplifier 32 is switched from the drive mode to the shutdown mode. Thus, the amplifier 32 is set in a quiescent state during the period the third output signal So3 is being acquired. Consequently, the amplifier 32 consumes less power than when the amplifier 32 remains operating in the drive mode.

The above steps allows the N number of output signals So1, So2, and So3 to be acquired in the order of So1→So2→So3.

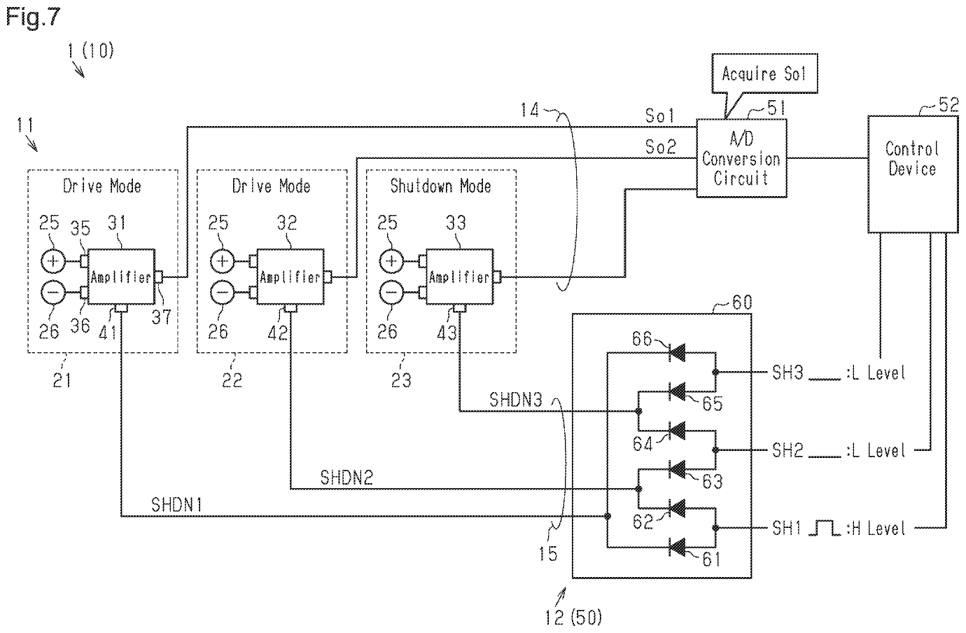

Then, referring to FIG. 7, in accordance with the acquisition order of the output signals So1, So2, and So3, the control device 52 shifts the first, or (N+1)th, control signal SH1 from the low level to a high level. When receiving the high-level control signal SH1, the shutdown signal generation circuit 60 outputs the shutdown signal SHDN1 at a high level and outputs the shutdown signal SHDN2 at a high level. This continues to operate the amplifier 31 that receives the high-level shutdown signal SHDN1 in the drive mode and switches the amplifier 32 that receives the high-level shutdown signal SHDN2 to the drive mode. In this state, the first amplifier 31 has been switched together with the third amplifier 33 to the drive mode when the amplifier 33 was switched to the drive mode (refer to FIG. 6). Thus, the amplifier 31 is already operating in the drive mode before the control signal SH1 shifts from the low level to the high level. After the first control signal SH1 shifts to the high level, the amplifier 31 operates in the drive mode without waiting for the switching time to allow for switching from the shutdown mode to the drive mode.

Then, the amplifier 31, which is operating in the drive mode, continues to output the output signal So1 to the A/D conversion circuit 51. The amplifier 32, which has been switched to the drive mode, amplifies the myogenic potential measured by the biological electrodes 25 and 26 to generate the output signal So2 and outputs the output signal So2 to the A/D conversion circuit 51. Further, among the output signals So1 and So2, the A/D conversion circuit 51 acquires the output signal So1 that is the (N+1)th (first) one in the acquisition order. For example, in response to the high level control signal SH1, the A/D conversion circuit 51 acquires the output signal So1 at a given sampling rate. The A/D conversion circuit 51 converts the acquired output signal So1 (analog signal) into a digital signal and outputs the converted digital signal to the control device 52. The control device 52, for example, performs a given analyzing processing on the digital signal received from the A/D conversion circuit 51. This acquires biological information (here, myoelectric signal) as a digital signal at the part where the first measurement portion 21 is attached.

In the step illustrated in FIG. 7, in response to acquisition of the output signal So3, the control device 52 shifts the third control signal SH3 from the high level to a low level. For example, after the A/D conversion circuit 51 completes acquisition of the output signal So3, the control device 52 shifts the control signal SH3 to the low level. Thus, the control signals SH2 and SH3 both have a low level. This shifts the shutdown signal SHDN3 to the low level. In response to the low-level shutdown signal SHDN3, the amplifier 33 is switched from the drive mode to the shutdown mode. Thus, the amplifier 33 is set in a quiescent state during the period the first output signal So1 is being acquired. Consequently, the amplifier 33 consumes less power than when the amplifier 33 remains operating in the drive mode.

Then, the steps illustrated in FIGS. 5 to 7 are repetitively performed to sequentially acquire the N number of output signals So1, So2, and So3 in the order of So1→So2→So3→So1→So2→So3 . . . .

The method for measuring biological information in the present embodiment sets the amplifiers 30 to the shutdown mode in response to acquisition of the corresponding output signals So. This allows the amplifiers 30 to be set in the shutdown mode during a period in which the corresponding output signals So are not being acquired. As a result, power consumption is reduced in the amplifiers 30. This reduces power consumption in the biological information measurement device 10. Further, in the present embodiment, the method for measuring biological information, when switching the one of the amplifiers 30 having an ordinal number of i in the acquisition order to the drive mode, where i is a natural number of 1 or greater, the amplifier 30 having the next ordinal number of (i+1) in the acquisition order is also switched to the drive mode. This starts switching the (i+1)th amplifier 30, which acquires the next output signal So, to the drive mode at an early stage and reduces the time for switching the (i+1)th amplifier 30 to the drive mode.

The present embodiment has the advantages described below.

(1) The biological information measurement device 10 includes the sensor unit 11, which includes the N number of (e.g., three) measurement portions 20, and the controller 50, which acquires the N number of output signals So from the N number of measurement portions 20 in a given acquisition order. Each of the N number of measurement portions 20 includes the biological electrodes 25 and 26 and the amplifier 30, which amplifies the biological information measured by the biological electrodes 25 and 26 to generate the output signal So. In response to a high-level shutdown signal SHDN, the amplifier 30 operates in a drive mode and generates the output signal So. In response to a low-level shutdown signal SHDN, the amplifier 30 operates in a shutdown mode and suspends generation of the output signal So. The controller 50 sequentially outputs the high-level shutdown signals SHDN to the N number of measurement portions 20 in accordance with the acquisition order of the N number of output signals So. The controller 50 outputs a low-level shutdown signal SHDN to each of the N number of measurement portions 20 in response to acquisition of the corresponding output signals So.

With this configuration, after acquiring the corresponding output signal So, the amplifier 30 is switched to the shutdown mode. This allows the amplifier 30 to be set in the quiescent state when the corresponding output signal So is not being acquired. Consequently, the amplifier 30 consumes less power than when the amplifier 30 is constantly operating in the drive mode. This reduces power consumption in the biological information measurement device 10.

(2) Power consumption of the biological information measurement device 10 may be reduced by driving only the amplifier 30 corresponding to the output signal So acquired by the controller 50 and setting the other amplifiers 30 in the shutdown mode. In this case, when acquiring the first output signal So1, only the first amplifier 31 is operated in the drive mode, and the other amplifier 32 and 33 are all set in the shutdown mode. When acquiring the second output signal So2, only the second amplifier 32 is operated in the drive mode, and the other amplifiers 31 and 33 are all set in the shutdown mode. When acquiring the third output signal So3, only the third amplifier 33 is operated in the drive mode, and the other amplifiers 31 and 32 are all set in the shutdown mode. In this case, if the sampling rate of the controller 50 becomes high and the time for switching the operating mode of the amplifiers 30 becomes longer, the amplifiers 30 may not be able to switch to the drive mode before the time at which the output signals So are to be acquired. This will hinder acquisition of the output signals So at the desired time.

In this respect, when outputting a high-level shutdown signal SHDN to the measurement portion 20 that is the ith one in the acquisition order, the controller 50 of the present embodiment outputs a high-level shutdown signal SHDN to the measurement portion 20 that is the (i+1)th one in the acquisition order. Thus, when switching the amplifier 30 that is the ith one in the acquisition order to the drive mode, the amplifier 30 that is the (i+1)th one in the acquisition order is also switched to the drive mode. For example, when the amplifier 31 that is the first one in the acquisition order is switched to the drive mode, the amplifier 32 that is the second one in the acquisition order is also switched to the drive mode. This starts switching the second amplifier 32, which acquires the next output signal So2, to the drive mode at an early stage and reduces the time for switching the second, or (i+1)th, amplifier 32 to the drive mode. As a result, even if the sampling rate of the controller 50 becomes high and the time for switching the operating mode of the amplifiers 30 becomes longer, the amplifiers 30 will have been switched to the drive mode at the time when the output signals So are to be acquired. This allows the output signals So to be acquired at the desired sampling rate, while reducing power consumption by switching the amplifiers 30 to the shutdown mode.

(3) The controller 50 includes the shutdown signal generation circuit 60 that generates the shutdown signals SHDN1, SHDN2, and SHDN3. The shutdown signal generation circuit 60 includes the diodes 61 and 62, the anode terminals of which receive the first control signal SH1, and the diodes 63 and 64, of which the anode terminals receive the second control signal SH2. The shutdown signal generation circuit 60 also includes the diodes 65 and 66, the anode terminals of which receive the third control signal SH3. The cathode terminals of the diodes 61 and 66 are connected to the first shutdown terminal 41 of the amplifier 31, and the cathode terminals of the diodes 62 and 63 are connected to the second shutdown terminal 42 of the amplifier 32. The cathode terminals of the diodes 64 and 65 are connected to the shutdown terminal 43 of the third amplifier 33. In this manner, the shutdown signal generation circuit 60 may be formed by only an (N×2) number, for example, six, diodes 61 to 66. This simplifies the configuration of the shutdown signal generation circuit 60.

### OTHER EMBODIMENTS

The above-described embodiment may be modified as described below. The above embodiment and the modified examples described below may be combined as long as there is no technical contradiction.

In the biological information measurement system 1 of the above embodiment, the communication performed between the control unit 12 and the information management device 80 is wireless communication. However, there is no limitation to wireless communication. For example, the communication performed between the control unit 12 and the information management device 80 may be wired communication.

In the above embodiment, the communication performed between the sensor unit 11 and the control unit 12 is wired communication using the first signal line 14 and the second signal line 15. However, there is no limitation to wired communication. For example, the communication performed between the sensor unit 11 and the control unit 12 may be wireless communication.

In the biological information measurement device 10 of the above embodiment, the sensor unit 11 and the control unit 12 are attached to the body B1, which is the subject of measurement. However, this is not a limitation. For example, among the sensor unit 11 and the control unit 12, only the sensor unit 11 may be attached to the body B1. In this case, the control unit 12 is not attached to the body B1 and is located at, for example, a position separated from the body B1.

In the above embodiment, the sensor unit 11 includes the three measurement portions 20. However, the number of the measurement portions 20 included in the sensor unit 11 is not limited to three and may be four or greater.

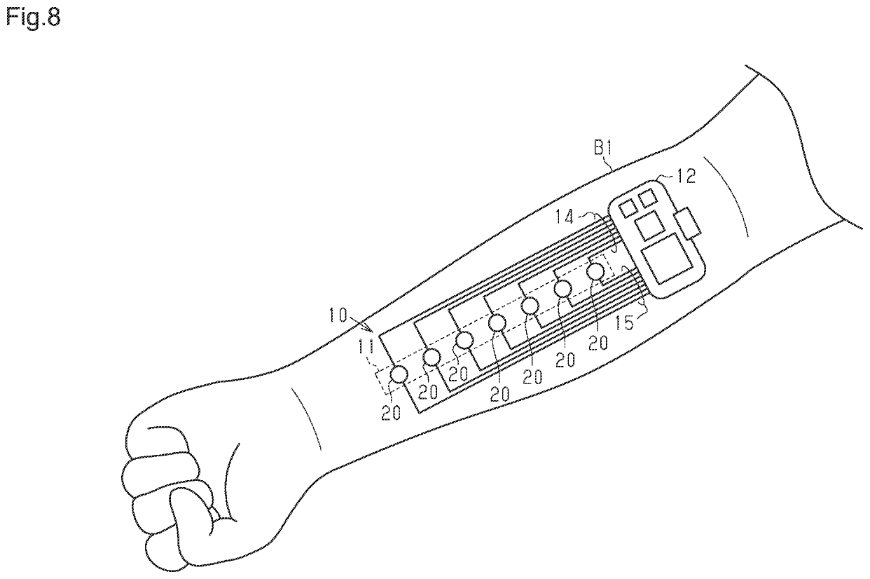

For example, as illustrated in FIG. 8, the sensor unit 11 may include seven measurement portions 20. In the sensor unit 11 of FIG. 8, the seven measurement portions 20 are arranged in a single line in the longitudinal direction of the subject's arm.

In the sensor unit 11 of the above embodiment, the N number of measurement portions 20 are arranged in a single line. However, there is no particular limitation to the arrangement of the N number of measurement portions 20.

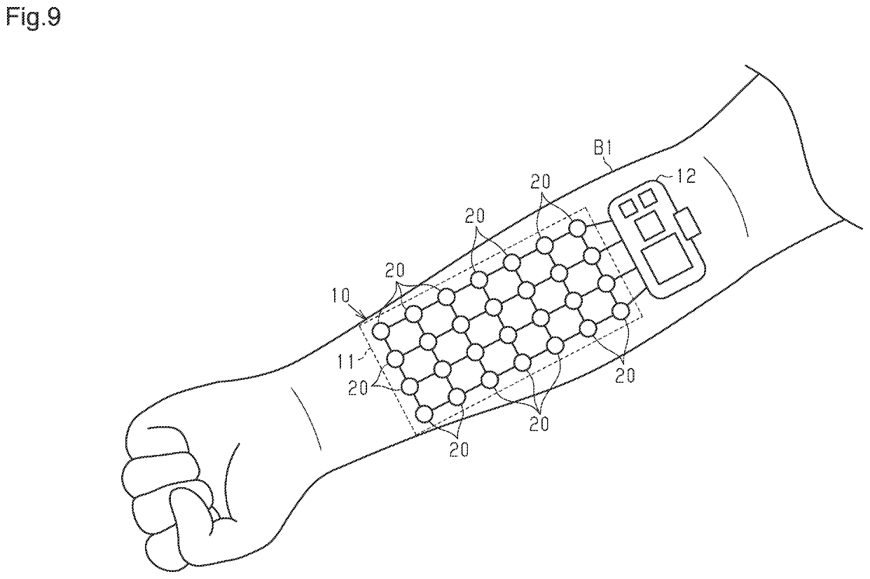

For example, as illustrated in FIG. 9, the N number of measurement portions 20 may be arranged in a matrix. For example, the N number of measurement portions 20 may be arranged in the longitudinal direction of the subject's arm or in a direction perpendicular to the longitudinal direction.

In the sensor unit 11 of the above embodiment, the N number of measurement portions 20 are arranged in the first direction in an order corresponding to the acquisition order of the N number of output signals So. However, this is not a limitation. For example, the N number of measurement portions 20 may be arranged in an order that differs from the acquisition order of the N number of output signals So. For example, when the measurement portions 21, 22, and 23 are arranged in the order illustrated in FIG. 2, the N number of the output signals So1, So2, and So3 may be acquired by the A/D conversion circuit 51 in the order of So2→So1→So3→So2→So1→So3 . . . .

In the controller 50 of the above embodiment, the N number of the output signals So1, So2, and So3 is acquired in the order of So1→So2→So3→So1→So2→So3 . . . . However, there is no limitation to such an order. For example, the N number of the output signals So1, So2, and So3 may be acquired in the order of So1→So2→So3→So2→So1→So2→So3 . . . .

In the above embodiment, when outputting a high-level shutdown signal SHDN to the ith measurement portion 20, the controller 50 also outputs a high-level shutdown signal SHDN to the (i+1)th measurement portion 20. Namely, when switching the ith amplifier 30 to the drive mode, the (i+1)th amplifier 30 is also switched to the drive mode. In other words, two amplifiers 30 are switched together to the drive mode. However, this is not a limitation. For example, an M number of measurement portions 20 may be switched together to the drive mode, where M is a natural number of 2 to N−1. For example, when the sensor unit 11 includes four or more measurement portions 20, three measurement portions 20 may be switched together to the drive mode.

In the above embodiment, the subject of biological information measurement is the body B1 of a human but may be the body of an animal.

Various changes in form and details may be made to the examples above without departing from the spirit and scope of the claims and their equivalents. The examples are for the sake of description only, and not for purposes of limitation. Descriptions of features in each example are to be considered as being applicable to similar features or aspects in other examples. Suitable results may be achieved if sequences are performed in a different order, and/or if components in a described system, architecture, device, or circuit are combined differently, and/or replaced or supplemented by other components or their equivalents. The scope of the disclosure is not defined by the detailed description, but by the claims and their equivalents. All variations within the scope of the claims and their equivalents are included in the disclosure.

1. A biological information measurement device, comprising:
a sensor unit including an N number of measurement portions, where N is a natural number of 3 or greater; and
a controller configured to sequentially acquire an N number of output signals from the N number of measurement portions in a given acquisition order, wherein:
each of the N number of measurement portions includes a biological electrode and an amplifier, the amplifier being configured to amplify biological information measured by the biological electrode to generate the output signal;
the amplifier includes a shutdown terminal configured to receive a shutdown signal;
the amplifier is configured to operate in a drive mode to generate the output signal in response to the shutdown signal having a first level and operate in a shutdown mode to suspend generation of the output signal in response to the shutdown signal having a second level; and
the controller is configured to:
sequentially output the shutdown signal having the first level to the N number of measurement portions in accordance with the acquisition order of the N number of output signals;
output the shutdown signal having the second level to each of the N number of measurement portions in response to acquisition of a corresponding one of the output signals; and
when outputting the shutdown signal having the first level to the one of the measurement portions having an ordinal number of i in the acquisition order, where i is a natural number of 1 or greater, also output the shutdown signal having the first level to the one of the measurement portions having an ordinal number of (i+1) in the acquisition order.

2. The biological information measurement device in accordance with claim 1, wherein:
the N number of output signals include first to Nth output signals corresponding to the acquisition order; and
the controller is configured to:
repetitively acquire the first to Nth output signals sequentially in an order of the first output signal, the second output signal, . . . , the Nth output signal, the first output signal, the second output signal, . . . ;
when outputting the shutdown signal having the first level to the one of the measurement portions having an ordinal number of j in the acquisition order, where j is a natural number of 1 to N−1, also output the shutdown signal having the first level to the one of the measurement portions having an ordinal number of (j+1) in the acquisition order; and
when outputting the shutdown signal having the first level to the one of the measurement portions having an ordinal number of N in the acquisition order, also output the shutdown signal having the first level to the one of measurement portions that is first in the acquisition order.

3. The biological information measurement device in accordance with claim 2, wherein:
the controller includes a shutdown signal generation circuit configured to generate the shutdown signal;
the shutdown signal generation circuit includes:
a first diode and a second diode, each including an anode terminal configured to receive a first control signal that controls an operating mode of the amplifier of the one of the measurement portions having the ordinal number of j in the acquisition order, and
a third diode and a fourth diode, each including an anode terminal configured to receive a second control signal that controls an operating mode of the amplifier of the one of the measurement portions having the ordinal number of N in the acquisition order;
the first diode includes a cathode terminal connected to the shutdown terminal of the amplifier of the one of the measurement portions having the ordinal number of j in the acquisition order;
the second diode includes a cathode terminal connected to the shutdown terminal of the amplifier of the one of the measurement portions having the ordinal number of (j+1) in the acquisition order;
the third diode includes a cathode terminal connected to the shutdown terminal of the amplifier of the one of the measurement portions having the ordinal number of N in the acquisition order; and
the fourth diode includes a cathode terminal connected to the shutdown terminal of the amplifier of the one of the measurement portions that is first in the acquisition order.

4. The biological information measurement device in accordance with claim 1, wherein the controller includes:
an A/D conversion circuit configured to sequentially acquire the N number of output signals and convert each of the N number of output signals that are analog signals into a digital signal; and
a control device configured to perform an analyzing processing on the digital signal generated by the A/D conversion circuit.

5. The biological information measurement device in accordance with claim 1, wherein the N number of measurement portions are sequentially arranged in a first direction in an order corresponding to the acquisition order.

6. The biological information measurement device in accordance with claim 1, further comprising:
a first signal line connecting an output terminal of the amplifier of each of the N number of measurement portions to the controller to transmit a corresponding one of the output signals; and
a second signal line connecting the shutdown terminal of the amplifier of each of the N number of measurement portions to the controller to transmit the shutdown signal.

7. A biological information measuring method, comprising:
sequentially acquiring an N number of output signals from an N number of measurement portions in a given acquisition order, where N is a natural number of 3 or greater;
sequentially outputting a shutdown signal having a first level to the N number of measurement portions in accordance with the acquisition order of the N number of output signals to set an operating mode of an amplifier of the one of the N number of measurement portions acquiring the shutdown signal of the first level to a drive mode that generates the output signal; and
outputting the shutdown signal having a second level to the one of the measurement portions having an ordinal number of i in the acquisition order, where i is a natural number of 1 or greater, in response to acquisition of the output signal from the ith one of the measurement portions to set the operating mode of the amplifier of the ith one of the measurement portions to a shutdown mode that suspends generation of the output signal;
wherein the sequentially outputting a shutdown signal having a first level includes, when outputting the shutdown signal having the first level to the one of the measurement portions having an ordinal number of i in the acquisition order, also outputting the shutdown signal having the first level to the one of the measurement portions having an ordinal number of (i+1) in the acquisition order.

8. The biological information measurement method in accordance with claim 7, wherein:
the N number of output signals include first to Nth output signals corresponding to the acquisition order;
the sequentially acquiring an N number of output signals includes repetitively acquiring the first to Nth output signals sequentially in an order of the first output signal, the second output signal, . . . , the Nth output signal, the first output signal, the second output signal, . . . ; and
the sequentially outputting a shutdown signal having a first level includes:
when outputting the shutdown signal having the first level to the one of the measurement portions having an ordinal number of j in the acquisition order, where j is a natural number of 1 to N−1, also outputting the shutdown signal having the first level to the one of the measurement portions having an ordinal number of (j+1) in the acquisition order, and
when outputting the shutdown signal having the first level to the one of the measurement portions having an ordinal number of N in the acquisition order, also outputting the shutdown signal having the first level to the one of the measurement portions that is first in the acquisition order.
